SEQUENCE LISTING (5 sequences)

SEQ ID NO: 1 - DNA, 22 bp, artificial
  forward primer
ctgaggtcaggagttcgagacc

SEQ ID NO: 2 - DNA, 18 bp, artificial
  reverse primer
ccacgcccggctaatttt

SEQ ID NO: 3 - DNA, 20 bp, artificial
  forward primer
gcggtggctcacgcctgtaa

SEQ ID NO: 4 - DNA, 20 bp, artificial
  reverse primer
ggagtgcagtggcgcgatct

SEQ ID NO: 5 - DNA, 23 bp, artificial sequence
  probe
cctggccaacatggtgaaacccc

(19) United States
(12) Patent Application Publication
(10) Pub. No.: US 2017/0159127 A1
(43) Pub. Date: Jun. 8, 2017

(54) AN IN VITRO DIAGNOSTIC METHOD OF CANCER

(71) Applicant: Euroclone S.p.A., Pero, Milano (IT)

(72) Inventors: Andrea Saccani, Milano (IT); Marco Agostini, Mestrino (IT); Chiara Bedin, Camisano Vicentino (IT)

(21) Appl. No.: 15/305,764

(22) PCT Filed: Apr. 24, 2014
(86) PCT No.: PCT/EP2014/058356

§ 371 Date: Oct. 21, 2016

(51) Int. Cl. C12Q 1/68

(52) U.S. Cl. CPC C12Q 1/6886; C12Q 2600/158; C12Q 2600/118

(57) ABSTRACT

The present invention relates to an in vitro method for the diagnosis, prognosis and treatment of cancer through the analysis in a body fluid sample of the total circulating DNA and the amount of DNA released by non-apoptic cells.

The present invention relates to an in vitro method for the diagnosis of cancer.

It is known that free DNA and DNA sequences can be found in body fluids as a result of the apoptosis process.

Differently from the sequences released by normal cells, there can be also found altered sequences which are indicative of the presence of altered, i.e. tumoral, cells.

The identification and possibly the quantification of such sequences, therefore, represents a way to identify the presence of a cancer in a subject and therefore a potentially powerful diagnostic tool.

### SUMMARY OF THE INVENTION

The present invention discloses a method for the in vitro diagnosis of cancer, wherein specific ALU sequences are detected in the a body sample.

### OBJECT OF THE INVENTION

A first object of the invention is represented by an in vitro method for the diagnosis of pathologies. According to a preferred aspect, said pathologies comprise the cancer and, in particular, the prostate cancer.

According to a particular aspect of the invention, it is disclosed a method of treatment comprising the step of performing the in vitro diagnostic method of the invention.

In a further aspect, the invention discloses a method for the prognosis of pathologies.

In a further object of the invention, it is disclosed a kit for the in vitro diagnosis of pathologies.

### DETAILED DESCRIPTION OF THE INVENTION

Definitions

For the purposes of the present invention, a sample is an isolated aliquot of a body fluid of a subject.

In particular, the sample used is a sample of plasma, serum, saliva, urine, fluid from lymph nodes or lymph organs, bone marrow, fluid from pleura, breast ducts, lacrimal fluid, serous fluid, peritoneal fluid, liquor (cerebral spinal fluid), stool, sputum, ascites, gastric or pancreatic juice, sweat.

In a preferred embodiment of the invention the sample is an isolated sample of blood or serum or liquor sample, whereas in a most preferred sample is an isolated sample of blood or serum.

The volume of the sample suitable for performing the method of the invention depends upon the type of the body fluid.

In particular, a suitable volume is comprised between about 300 μl-1 ml and preferably is about 500 μl.

In a first aspect of the invention, the blood sample is not purified.

Accordingly, once taken from the subject, the only steps performed comprise the centrifugation and/or filtration in order to remove the particulate (cells).

In addition, other suitable treatments can be performed on the sample in order to remove or inactivate proteins, like for instance a step of treatment with enzymes.

In another embodiment of the invention, the sample is subjected to a step for the DNA extraction.

Said step can be performed according to well-known techniques in the art, like for instance the extraction with phenol/chloroform.

For the purposes of the present invention, a subject is intended to be a human or an animal.

Within animals, pets and courtyard animals are preferred, wherein mammals are particularly preferred.

In an even preferred embodiments, animals refers to dogs.

The “total circulating DNA” according to the invention can be determined with quantitative PCR or other method suitable in the art.

Within the present invention, the “amount of” is used to refer to a value which is representative of the concentration of a particular sequence (nucleic sequence) in the sample.

“Representative” is used to refer to a relation between a two values.

For instance, a measurement of a compound in a sample could provide a value which can be used to determine the concentration through a known calculation.

The way to calculate the concentration from the first value depends upon the circumstances as well as upon the method used.

The “DNA integrity” refers to the ratio of the amount of the circulating DNA released from cancer cells to the total amount of DNA.

In the diagnostic method of the invention, the subject is a person or an animal likely to develop cancer or another cancer (i.e. recurrence of the cancer) or suspected of having cancer.

The in vitro diagnostic method of the present invention comprises in particular the steps of:

1) determining the total circulating DNA in a sample;

2) determining the DNA released by cancer cells;

3) determining the value “II” (Integrity Index), which corresponds to the ratio between the values of steps 1) and 2).

In particular, the step 1) comprises determining the amount of ALU 83 present in the sample.

For said purpose, the following primers are used:

| Sequence 5′→3′: | | SEQ. ID. n. |
|---|---|---|
| forward | CTGAGGTCAGGAGTTCGAGACC | 1 |
| reverse | CCACGCCCGGCTAATTTT | 2 |

As for step 2), it comprises determining the amount of ALU 244 present in the sample.

For said purpose, the following primers are used:

| Sequence 5′→3′: | | SEQ. ID. n. |
|---|---|---|
| forward | GCGGTGGCTCACGCCTGTAA | 3 |
| reverse | GGAGTGCAGTGGCGCGATCT | 4 |

In step 3) the ratio “II” between the amount of the DNA released by cancer cells and the total circulating DNA is calculated, as it can be represented below:

$$II = \frac{ALU 244 value}{ALU 83 value}$$

In a preferred embodiment “value” stands for a measure of quantity, like concentration, determined with quantitative PCR; therefore, the “C” value is calculated as below:

$$C = \frac{ALU 244 - qPCR value}{ALU 83 - qPCR value}$$

According to a preferred embodiment of the present invention, in the above steps 1) and 2) the ALU sequences are amplified by quantitative PCR (qPCR).

In addition to the above steps 1) to 3), the in vitro diagnostic method of the invention comprises the additional step of comparing the data obtained for the “II” value with a pool of reference values.

For the purposes of the present invention a pool of reference values comprises values which are known to correspond to either:
health patients, or
patients with cancer, or
to a disease state of a group of patients.

In particular, the reference value depends upon characteristics of the patient undergoing the in vitro diagnostic method of the invention, like age, sex, race, etc.

In addition to that, the reference value depends upon the kind of sample under examination and upon the type of tumor which is investigated.

In the preferred embodiment, the method above disclosed allows the diagnosis of the prostate cancer.

According to another object of the invention, there is disclosed a method of treatment comprising the step of performing the in vitro diagnostic method of the invention.

In particular, said method can be used for the treatment of cancer and, more in particular, of the prostate cancer.

More in detail, said method can be performed on a subject who developed a cancer or who is being treated against a cancer or who underwent treatment against cancer, like a surgical intervention to remove cancer.

Accordingly, the present method can be used for monitoring the progression of the cancer as well as the response to the therapeutic treatment.

In fact, any variation in the data collected can be indicative of the progression or regression of the cancer in case the therapeutic treatment is ineffective or successful.

Therefore, the method of the invention can also be used for identifying if a drug or a treatment is effective against cancer.

In another object of the invention, there is disclosed a method for the prognosis of the cancer comprising carrying out the method of the invention.

For said purpose, the method can be performed on a subject which is likely to develop a cancer in the future.

In this regard, consideration shall be done on the environment the subject lives in (whether in the past there has been contact with potentially dangerous substances) and/or on their familiarity with the pathology (for instance, if one or more close relatives developed a cancer).

The data of the “C” value can be collected at one or subsequent times and compared to reference values, which are indicative of the onset of the pathology.

Also in this case, the reference data depends upon characteristics of the patient undergoing the method of the invention, like age, sex, race, etc.

In addition to that, the reference data depends upon the kind of sample under examination and upon the type of tumor which is investigated.

Thanks to the method of the invention, the curative treatment of the patient can be advantageously started at a very early stage of the pathology.

The methods above disclosed which make use of the in vitro diagnostic method of the invention can be used in combination with other method so as to achieve even more reliable and effective results.

In accordance with a further object of the present invention, it is provided a kit for performing the in vitro diagnostic method of the invention.

In particular, said kit comprises:
primers for the amplification of ALU 83; and
primers for the amplification of ALU 244.

More in detail, each of the primers is included in an appropriate reaction solution comprising a suitable probe.

In a first embodiment of the invention, a reactant for amplification of ALU83 comprises:

| | | |
|---|---|---|
| Fluocycle II ™ Master Mix probe (without | 10 | μl |
| ROX) (Euroclone S.p.A.) | | |
| ALU83 fwd (10 μM) | 0.1 | μl |
| ALU83 rev (10 μM) | 1.8 | μl |
| probe (10 μM) | 0.5 | μl |
| water | to 20 | μl |

Fluocycle II™ Master Mix formulation comprises: 100 mM KCl, 20 mM Tris HCl pH 8.3, 0.02% Tween-20, 0.8 mM of each dNTPs (dATP, dCTP, dGTP, dTTP), 200 units/ml Taq DNA polymerase, 8 mM MgCl₂, stabilizers.

On the other side, the reactant for amplification of ALU244 comprises:

| | | |
|---|---|---|
| Fluocycle II ™ Master Mix probe (without | 10 | μl |
| ROX) (Euroclone S.p.A.) | | |
| ALU244 fwd (10 μM) | 1.8 | μl |
| ALU244 rev (10 μM) | 1.8 | μl |
| probe (10 μM) | 0.5 | μl |
| water | to 20 | μl |

Fluocycle II™ Master Mix formulation comprises: 100 mM KCl, 20 mM Tris HCl pH 8.3, 0.02% Tween-20, 0.8 mM of each dNTPs (dATP, dCTP, dGTP, dTTP), 200 units/ml Taq DNA polymerase, 8 mM MgCl₂, stabilizers.

The kit of the invention preferably also comprises a sequence for performing the standard reference curve.

For instance, a preparation of an ALU sequence, which can be in a lyophilized form or in solution.

According to another aspect, the kit may comprise in addition a sample which can be used to verify the inter-assay variability.

In a first kit of the invention, there are included:
the above reactant for amplification of ALU83;
the above reactant for amplification of ALU244;
the sequence for performing the standard reference curve, such as a preparation of an ALU sequence;
a sample for verifying the inter-assay variability.

In an alternative kit, there can be included:
a solution Fluocycle II™ Master Mix probe (without ROX) (Euroclone S.p.A.);
a solution comprising the primers for the ALU83 sequence and a suitable probe;
a solution comprising the primers for the ALU244 sequence and a suitable probe;
the sequence for performing the standard reference curve, such as a preparation of an ALU sequence;
a reference sample for verifying the inter-assay variability.

In the above kit, the primer solutions for the ALU sequences preferably are:

| | |
|---|---|
| ALU83 fwd (10 μM) | 0.1 μl |
| ALU83 rev (10 μM) | 1.8 μl |
| probe (10 μM) | 0.5 μl |
| water | |
| and | |
| ALU244 fwd (10 μM) | 1.8 μl |
| ALU244 rev (10 μM) | 1.8 μl |
| probe (10 μM) | 0.5 μl |
| water | |

The kit can further comprise a collection of data that can be used as a reference in the in vitro diagnostic, therapeutic or prognostic method of the invention.

Materials and Methods

cfDNA Extraction Kit

QIAamp(R) UltraSens™ Virus Kit (QIAGEN):

Starting volume plasma: 500 μl

cfDNA elution volume: 50 μl

Real Time PCR Amplification Protocol

### Standard Curve

| | | |
|---|---|---|
| Standard 6: | 0.5 | pg/μl |
| Standard 5: | 1 | pg/μl |
| Standard 4: | 10 | pg/μl |
| Standard 3: | 100 | pg/μl |
| Standard 2: | 1 * 10³ | pg/μl |
| Standard 1: | 10 * 10³ | pg/μl |

| reactant for mix ALU83 | μl |
|---|---|
| Fluocycle II Master Mix probe (NO ROX) | 10 |
| ALU83fwd (10 μM) | 0.1 |
| ALU83rev (10 μM) | 1.8 |
| probe (10 μM) | 0.5 |
| H₂O | 6.6 |
| cfDNA/Standard | 1 |
| volume tot | 20 |

| reactant for mix ALU244 | μl |
|---|---|
| Fluocycle II Master Mix probe (NO ROX) | 10 |
| ALU244fwd (10 μM) | 1.8 |
| ALU244rev (10 μM) | 1.8 |
| probe (10 μM) | 0.5 |
| H₂O | 4.9 |
| cfDNA/Standard | 1 |
| volume tot | 20 |

Amplification Protocol for the ALU Mix

| | | |
|---|---|---|
| 95° C. | 5 min | |
| 95° C. | 15 sec | X40 cycles |
| 62° C. | 1 min | |

Probe

SEQ.ID. n.5
6FAM-CCTGGCCAACATGGTGAAACCCC-TMR

### EXAMPLE 1

### Quantitative PCR of Plasma DNA Fragments

The quantification of the cell free DNA (cf-DNA) fragments in plasma was performed by quantitative real time PCR (qPCR), which amplified and quantified the shortest and longest DNA fragments. To maximize the sensitivity of cf-DNA quantification, the ALU repeats were used as a target of the qPCR.

Two primer pairs were designed as follows: the first primer set (ALU244) was amplified only the longer (244 bp) DNA fragment, whereas the second primer set (ALU83) were amplified both the shorter (83 bp) and longer, because the ALU83 annealing site was within the ALU244 annealing site. The results obtained using the ALU83 primers represent the total cell free plasma circulating DNA, while the results obtained using the ALU244 primers reflect the amount of DNA released from non-apoptotic cells.

The probe sequence was 6FAM-CCTGGCCAACATGGTGAAACCCC-TMR.

The two reactions were performed in 20 μl final volume containing 1 μl of sample, 1× FluoCycle™ II Probe (Euroclone®, Milan, Italy), 0.25 μM of probe and 0.9 μM of each ALU244 primer in ones or 0.05 μM of forward and 0.9 μM of reverse primer for ALU83 in the other. The qPCR amplification consisted of an initial denaturation step for 5 minutes at 95° C., followed by 40 cycles of denaturation for 15 seconds at 95° C. and annealing/extension for 1 minute at 62° C., using the 7300 Real Time PCR System and 7500 Fast Real Time PCR System (Applied Biosystems, Milan, Italy).

The samples were tested in triplicate and an appropriate negative reference (no template) samples were included.

The absolute amount of cf-DNA in each sample was determined by a standard curve, using 10-fold serial dilutions (from 10 ng to 1 pg) of genomic DNA obtained from healthy subject buffy-coat.

1. An in vitro diagnostic method comprising the step of determining the amount of the ALU 244 sequence in an isolated sample of body fluid and the step of determining the amount of the ALU 83 sequence in the isolated sample of body fluid and the step of determining a value which is the ratio between the amount of ALU 244 and of ALU 83 in the isolated sample of body fluid.

2. (canceled)

3. The in vitro diagnostic method according to claim 1, wherein the isolated sample of body fluid is purified in a purification step before determining the amount of the ALU sequence.

4. (canceled)

5. The in vitro diagnostic method according to claim 1, wherein the isolated sample of the body fluid is treated in order to extract the DNA from the isolated sample before determining the amount of the ALU sequence.

6. (canceled)

7. The in vitro diagnostic method according to claim 1, wherein the amount of ALU 244 sequences is indicative of the DNA released by tumoral cells.

8. The in vitro diagnostic method according to claim 2, wherein the amount of ALU 83 sequences is indicative of the total circulating DNA.

9. (canceled)

10. The in vitro diagnostic method according to claim 1, wherein said ratio is compared to a reference value from health patients.

11. The in vitro diagnostic method according to claim 1, wherein said isolated sample of body fluid is a sample of blood, plasma, serum, saliva, urine, fluid from lymph nodes or lymph organs, bone marrow, fluid from pleura, breast ducts, lacrimal fluid, serous fluid, peritoneal fluid, cerebral spinal fluid, stool, sputum, ascites, gastric or pancreatic juice, sweat.

12. (canceled)

13. The in vitro diagnostic method according to claim 1, wherein said body fluid is from a human or an animal.

14. The in vitro diagnostic method according to claim 1, wherein said body fluid is from dog.

15. The in vitro diagnostic method according to claim 1, wherein said isolated sample has a volume comprised between about 300 μl-1 ml and preferably is about 500 μl.

16. The in vitro diagnostic method according to claim 1, wherein the step of determining the amount of ALU 244 comprises the use of the following primers:

| Sequence 5′→3′: | | |
|---|---|---|
| forward | GCGGTGGCTCACGCCTGTAA | SEQ.ID. n.1 |
| reverse | GGAGTGCAGTGGCGCGATCT | SEQ.ID. n.2 |

17. The in vitro diagnostic method according to claim 1, wherein the step of determining the amount of ALU 83 comprises the use of the following primers:
forward
SEQ.ID. n. 3
CTGAGGTCAGGAGTTCGAGACC
reverse
SEQ.ID. n. 4
CCACGCCCGGCTAATTTT

18. The in vitro diagnostic method according to claim 1, wherein the amount of ALU 244 and/or of ALU 83 is determined by quantitative real-time polymerase chain reaction.

19. The in vitro diagnostic method according to claim 18, wherein said amplification step comprises a denaturation step for 5 minutes at 95° C., followed by 40 cycles of denaturation for 15 seconds at 95° C. and annealing/extension for 1 minute at 62° C.

20. The in vitro diagnostic method according to claim 1, wherein said cancer is prostate cancer.

21. (canceled)

22. A method for the treatment or for the prognosis of cancer comprising the step of performing the in vitro diagnostic method of claim 1.

23. An isolated nucleotidic sequence having a sequence selected from the group comprising SEQ.ID. n. 1, 2, 3 4.

24. A kit for performing a diagnostic method or a prognostic method or a therapeutic method comprising any one of the isolated sequences of claim 23.

25. The kit according to claim 24, wherein any of said sequences is comprised in a preparation further comprising a suitable amplification solution.

26. The kit according to claim 24, wherein said suitable amplification solution is Fluorocycle II Master Mix (Euroclone S.p.A.).

27. The kit according to claim 24, further comprising a probe.

28. The kit according to claim 27, wherein said probe is:
SEQ.ID. n. 5.
6FAM-CCTGGCCAACATGGTGAAACCCC-TMR

29. The kit according to claim 28, further comprising a reference sample for verifying the inter-assay variability.

30-33. (canceled)
